SEQUENCE LISTING (12 sequences)

SEQ ID NO: 1 - DNA, 603 bp, Artificial Sequence
  CDO cds
  misc_feature (1)..(603)
atggaacgcaccgaactgctgaaaccgcgtaccttggctgatctgattcggattctgcatgaactgtttgccggtgatgaggtcaatgttgaggaagtgcaagcagttctggaggcgtatgaaagcaatccggcagaatgggcgttatacgccaaatttgaccagtatcgctatacgcgtaatctggtagatcagggtaatggcaaattcaacctgatgatcttatgctggggagaaggtcatgggagtagcattcacgatcataccgatagccattgctttctgaaactcttgcaaggcaatctgaaagaaaccctctttgattggccggataagaaatccaacgaaatgatcaagaaatctgaacgtactcttcgcgaaaatcagtgtgcgtacatcaacgactccattggtttgcaccgcgtagagaacgtcagccataccgaaccagctgtgtcactgcacctttactctcctccgtttgacacgtgtcatgcctttgaccagcgtacaggccacaagaacaaagtgacgatgaccttccactcgaaattcgggattcgcacacccttcacgactagtggctcgttagagaacaactaa

SEQ ID NO: 2 - protein, 200 aa, Artificial Sequence
  CDO Protein
  PEPTIDE (1)..(200)
MERTELLKPRTLADLIRILHELFAGDEVNVEEVQAVLEAYESNPAEWALYAKFDQYRYTRNLVDQGNGKFNLMILCWGEGHGSSIHDHTDSHCFLKLLQGNLKETLFDWPDKKSNEMIKKSERTLRENQCAYINDSIGLHRVENVSHTEPAVSLHLYSPPFDTCHAFDQRTGHKNKVTMTFHSKFGIRTPFTTSGSLENN

SEQ ID NO: 3 - DNA, 1842 bp, Artificial Sequence
  CSAD cds rrnB Terminator
  misc_feature (1)..(1842): CSADhs-rrnB
atgattccgagcaagaagaatgcggttctggtggatggagtcgtgctcaatggaccgactacagatgccaaagcaggcgaaaagttcgtcgaagaggcctgtcgcctgatcatggaggaagtggtactcaaagccaccgatgttaacgaaaaagtttgtgaatggcgtcctccagaacagctgaaacagctgctggatctggaaatgcgcgattcgggtgaaccgccacacaaactgctcgagttgtgtcgtgacgtgatccattattccgtgaaaacgaatcatccgcgcttctttaaccagctctacgccggtttggactattacagcctcgttgctcgctttatgaccgaagccttaaatccgtcggtctacacctatgaggtaagcccggttttcctgctggtagaagaagcggtgttgaaaaagatgattgagttcattgggtggaaagaaggggatggcattttcaacccaggtggtagtgtcagcaacatgtatgcgatgaatttggcgcgctacaagtactgcccggacatcaaagagaaaggccttagtggcagtcctcgtctgatcctctttacttcggcggaatgccactacagcatgaaaaaagccgcgagctttctggggattgggacagaaaacgtgtgttttgtggaaactgacggtcgtggcaaaatgattcctgaagaactggaaaaacaggtgtggcaagcgcgcaaagaaggagcagctccctttctggtgtgtgcaacctccggcacgaccgttttgggcgcatttgatccccttgatgaaattgccgatatctgcgaacgccattccctgtggctgcatgttgacgcgtcttggggtggttcagctctgatgtctcgtaaacaccgcaaacttctgcatggcattcatcgggcagattccgttgcttggaatccgcacaaaatgctgatggctggtattcagtgctgtgcactgctggtgaaagacaaatctgacttactgaagaaatgctatagtgcaaaagcgtcttacttatttcagcaggataagttctacgatgtctcctatgataccggcgacaaatcgatccagtgctcacgtcgtccagatgccttcaaattctggatgacctggaaggcgttgggcacgttaggcctggaggaacgtgtgaatcgcgcgttagcgctgagtcgctatctggtagatgagatcaaaaaacgtgaaggctttaagttgctgatggaacctgagtatgcgaacatctgcttttggtatattccgccctcacttcgtgaaatggaggaaggtccggaattttgggccaaacttaaccttgtagccccggctattaaagagcggatgatgaaaaaaggctcattaatgctggggtatcaaccgcatcgcggtaaagtcaacttctttcgccaagtggtcattagcccgcaagtttcgcgcgaggatatggactttctgctggatgaaatcgatttactgggtaaggacatgtaatctagagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaac

SEQ ID NO: 4 - protein, 502 aa, Artificial Sequence
  CSAD Protein
  PEPTIDE (1)..(502)
MIPSKKNAVLVDGVVLNGPTTDAKAGEKFVEEACRLIMEEVVLKATDVNEKVCEWRPPEQLKQLLDLEMRDSGEPPHKLLELCRDVIHYSVKTNHPRFFNQLYAGLDYYSLVARFMTEALNPSVYTYEVSPVFLLVEEAVLKKMIEFIGWKEGDGIFNPGGSVSNMYAMNLARYKYCPDIKEKGLSGSPRLILFTSAECHYSMKKAASFLGIGTENVCFVETDGRGKMIPEELEKQVWQARKEGAAPFLVCATSGTTVLGAFDPLDEIADICERHSLWLHVDASWGGSALMSRKHRKLLHGIHRADSVAWNPHKMLMAGIQCCALLVKDKSDLLKKCYSAKASYLFQQDKFYDVSYDTGDKSIQCSRRPDAFKFWMTWKALGTLGLEERVNRALALSRYLVDEIKKREGFKLLMEPEYANICFWYIPPSLREMEEGPEFWAKLNLVAPAIKERMMKKGSLMLGYQPHRGKVNFFRQVVISPQVSREDMDFLLDEIDLLGKDM

SEQ ID NO: 5 - DNA, 65 bp, Artificial Sequence
  Primer
  primer_bind (1)..(65): cdorn-1f
gtaaattgatcaagtattctgactaagttaaggaggaaattatatggaacgcaccgaactgctga

SEQ ID NO: 6 - DNA, 57 bp, Artificial Sequence
  Primer
  primer_bind (1)..(57): csadhs-2r
cattcttcttgctcggaatcatataatttcctccttattagttgttctctaacgagc

SEQ ID NO: 7 - DNA, 57 bp, Artificial Sequence
  Primer
  primer_bind (1)..(57): csadhs-3f
gctcgttagagaacaactaataaggaggaaattatatgattccgagcaagaagaatg

SEQ ID NO: 8 - DNA, 55 bp, Artificial Sequence
  Primer
  primer_bind (1)..(55): glf-2r
acgcggcgcatctcgggcagcgttgggtcactagttgcttcgcaacgttcaaatc

SEQ ID NO: 9 - DNA, 2379 bp, Artificial Sequence
  ppsA-MHI cds
  misc_feature (1)..(2379)
atgtccaacaatggctcgtcaccgctggtgctttggtataaccaactcggcatgaatgatgtagacagggttgggggcaaaaatgcctccctgggtgaaatgattactaatctttccggaatgggtgtttccgttccgaatggtttcgccacaaccgccgacgcgtttaaccagtttctggaccaaagcggcgtaaaccagcgcatttatgaactgctggataaaacggatattgacgatgttactcagcttgcgaaagcgggcgcgcaaatccgccagtggattatcgacactcccttccagcctgagctggaaaacgccatccgcgaagcctatgcacagctttccgccgatgacgaaaacgcctcttttgcgatgcgctcctccgccaccgcagaagatatgccggacgcttcttttgccggtcagcaggaaaccttcctcaacgttcagggttttgacgccgttctcgtggcagtgaaacatgtatttgcttctctgtttaacgatcgcgccatctcttatcgtgtgcaccagggttacgatcaccgtggtgtggcgctctccgccggtgttcaacggatggtgcgctctgacctcgcatcatctggcgtgatgttctccattgataccgaatccggctttgaccaggtggtgtttatcacttccgcatggggccttggtgagatggtcgtgcagggtgcggttaacccggatgagttttacgtgcataaaccgacactggcggcgaatcgcccggctatcgtgcgccgcaccatggggtcgaaaaaaatccgcatggtttacgcgccgacccaggagcacggcaagcaggttaaaatcgaagacgtaccgcaggaacagcgtgacatcttctcgctgaccaacgaagaagtgcaggaactggcaaaacaggccgtacaaattgagaaacactacggtcgcccgatggatattgagtgggcgaaagatggccacaccggtaaactgttcattgtgcaggcgcgtccggaaaccgtgcgctcacgcggtcaggtcatggagcgttatacgctgcattcacagggtaagattatcgccgaaggccgtgctatcggtcatcgcatcggtgcgggtccggtgaaagtcatccatgacatcagcgaaatgaaccgcatcgaacctggcgacgtgctggttactgacatgaccgacccggactgggaaccgatcatgaagaaagcatctgccatcgtcaccaaccgtggcggtcgtacctgtcacgcggcgatcatcgctcatgaactgggcattccggcgatagtgggctgtggagatgcaacagaacggatgaaagacggtgagaacgtcactgtttcttgtgccgaaggtgataccggttacgtctatgcggagttgctggaatttagcgtgaaaagctccagcgtagaaacgatgccggatctgccgttgaaagtgatgatgaacgtcggtaacccggaccgtgctttcgacttcgcctgcctaccgaacgaaggcgtgggccttgcgcgtctggaatttatcatcaaccgtatgattggcgtccacccacgcgcactgcttgagtttgacgatcaggaaccgcagttgcaaaacgaaatccgcgagatgatgaaaggttttgattctccgcgtgaattttacgttggtcgtctgactgaagggatcgcgacgctgggtgccgcgttttatccgaagcgcgtcattgtccgtctctctgattttaaatcgaacgaatatgccaacctggtcggtggtgagcgttacgagccagatgaagagaacccgatgctcggcttccgtggcgcgggccgctatgtttccgacagcttccgcgactgtttcgcgctggagtgtgaagcagtgaaacgtgtgcgcaacgacatgggactgaccaacgttgagatcatgatcccgttcgtgcgtaccgtagatcaggcgaaagcggtggttgaagaactggcgcgtcaggggctgaaacgtggcgagaacgggctgaaaatcatcatgatgtgtgaaatcccgtccaacgccttgctggccgagcagttcctcgaatatttcgacggcttctcaattggctcaaacgatatgacgcagctggcgctcggtctggaccgtgactccggcgtggtgtctgaattgttcgatgagcgcaacgatgcggtgaaagcactgctgtcgatggctatccgtgccgcgaagaaacagggcaaatatgtcgggatttgcggtcagggtccgtccgaccacgaagactttgccgcatggttgatggaagaggggatcgatagcctgtctctgaacccggacaccgtggtgcaaacctggttaagcctggctgaactgaagaaataa

SEQ ID NO: 10 - protein, 792 aa, Artificial Sequence
  PpsA-MHI Protein
  PEPTIDE (1)..(792)
MSNNGSSPLVLWYNQLGMNDVDRVGGKNASLGEMITNLSGMGVSVPNGFATTADAFNQFLDQSGVNQRIYELLDKTDIDDVTQLAKAGAQIRQWIIDTPFQPELENAIREAYAQLSADDENASFAMRSSATAEDMPDASFAGQQETFLNVQGFDAVLVAVKHVFASLFNDRAISYRVHQGYDHRGVALSAGVQRMVRSDLASSGVMFSIDTESGFDQVVFITSAWGLGEMVVQGAVNPDEFYVHKPTLAANRPAIVRRTMGSKKIRMVYAPTQEHGKQVKIEDVPQEQRDIFSLTNEEVQELAKQAVQIEKHYGRPMDIEWAKDGHTGKLFIVQARPETVRSRGQVMERYTLHSQGKIIAEGRAIGHRIGAGPVKVIHDISEMNRIEPGDVLVTDMTDPDWEPIMKKASAIVTNRGGRTCHAAIIAHELGIPAIVGCGDATERMKDGENVTVSCAEGDTGYVYAELLEFSVKSSSVETMPDLPLKVMMNVGNPDRAFDFACLPNEGVGLARLEFIINRMIGVHPRALLEFDDQEPQLQNEIREMMKGFDSPREFYVGRLTEGIATLGAAFYPKRVIVRLSDFKSNEYANLVGGERYEPDEENPMLGFRGAGRYVSDSFRDCFALECEAVKRVRNDMGLTNVEIMIPFVRTVDQAKAVVEELARQGLKRGENGLKIIMMCEIPSNALLAEQFLEYFDGFSIGSNDMTQLALGLDRDSGVVSELFDERNDAVKALLSMAIRAAKKQGKYVGICGQGPSDHEDFAAWLMEEGIDSLSLNPDTVVQTWLSLAELKK

SEQ ID NO: 11 - DNA, 50 bp, Artificial Sequence
  Primer
  primer_bind (1)..(50)
  primer_bind (1)..(50): pps-9f
atgtccaacaatggctcgtcaccgctggtgcctcacgctgccgcaagcac

SEQ ID NO: 12 - DNA, 51 bp, Artificial Sequence
  Primer
  primer_bind (1)..(51)
  primer_bind (1)..(51): pps-10r
ttatttcttcagttcagccaggcttaaccaagaacaactgttcaccgttag

(19) United States
(12) Patent Application Publication
(10) Pub. No.: US 2023/0406817 A1
(43) Pub. Date: Dec. 21, 2023

(54) METHOD FOR ENZYMATIC OXIDATION OF SULFINIC ACIDS TO SULFONIC ACIDS

(71) Applicant: WACKER CHEMIE AG, Munich (DE)

(72) Inventor: Rupert PFALLER, Munich (DE)

(73) Assignee: Wacker Chemie AG, Munich (DE)

(21) Appl. No.: 18/035,610

(22) PCT Filed: Jul. 5, 2021
(86) PCT No.: PCT/EP2021/068556

§ 371 Date: May 5, 2023

(51) Int. Cl. C07C 303/16; C12P 13/00

(52) U.S. Cl. CPC C07C 303/16; C12P 13/001; C12R 2001/19

(57) ABSTRACT

A process for the enzymatic oxidation of sulfinic acids includes sulfinic acids of formula H₂N—CH(R)—CH₂—SO₂H to sulfonic acids of formula H₂N—CH(R)—CH₂—SO₃H and an enzyme selected from the class of H₂O₂-generating oxidases in the presence of the substrate of said enzyme.

The invention relates to a process for the enzymatic oxidation of sulfinic acids of formula H₂N—CH(R)—CH₂—SO₂H to sulfonic acids of formula H₂N—CH(R)—CH₂—SO₃H with an enzyme selected from the class of H₂O₂-generating oxidases in the presence of the substrate of said enzyme, especially for the enzymatic oxidation of L-cysteine sulfinic acid to L-cysteic acid and of hypotaurine to taurine.

Sulfinic acids are a class of chemical compounds containing organically bound sulfur and oxygen of the general structure R—S(═O)—OH, where R is an organic radical. The parent substance sulfinic acid has the structure H—S(═O)—OH and is tautomeric with sulfoxylic acid HO—S—OH. The salts of sulfinic acids are sulfinates.

Sulfonic acids are organic sulfur compounds having the general structure R—SO₂—OH, where R is an organic radical. Their salts and esters having the respective general structures R—SO₂—O⁻ and R₁—SO₂—O—R₂ (R₁, R₂ are in each case organic radicals) are known as sulfonates.

The conversion of sulfinic acids into sulfonic acids is chemically possible by oxidation, for example with peroxides (see for example Chauvin and Pratt, Angew. Chem. Int. Ed. (2017) 56: 6255-6259). A possible enzymatic oxidation of sulfinic acids to sulfonic acids is of no consequence in the chemical industry, but is of interest for biotechnological processes for the production of naturally occurring sulfonic acids from the corresponding sulfinic acids.

Examples of naturally occurring sulfinic acids include L-cysteine sulfinic acid and hypotaurine. Examples of naturally occurring sulfonic acids include L-cysteic acid and taurine.

Taurine (2-aminoethanesulfonic acid, CAS number 107-35-7) is an aminosulfonic acid that occurs naturally in nature as a breakdown product of the amino acids cysteine and methionine. Taurine is of economic importance; it is for example a constituent of energy drinks and is also used in pet food, for example for cats or in fish farming (Salze and Davis, Aquaculture (2015) 437: 215-229). Taurine is however considered to have health-promoting effects too (Ripps and Shen, Molecular Vision (2012) 18: 2673-2686).

Taurine for commercial use is currently produced chemically. One known process is for example that of Changshu Yudong Chemical Factory, which starts with ethylene and leads to taurine via ethyleneimine. With the consumer-driven trend away from chemically produced ingredients, biotechnological processes for the production of taurine are increasingly being investigated.

In nature, taurine occurs almost exclusively in the animal kingdom, there being only a few examples where it occurs in plants, algae or bacteria. There are various biosynthetic pathways to taurine (see for example the KEGG Pathway Database: “Taurine and hypotaurine metabolism”), starting inter alia from L-cysteine. The most important synthetic steps leading from L-cysteine to taurine are shown in equations (1) to (5).

L-Cysteine+O₂−>L-Cysteine sulfinic acid (1)

L-Cysteine sulfinic acid+½O₂−>L-Cysteic acid (2)

L-Cysteine sulfinic acid−>Hypotaurine+CO₂ (3)

Hypotaurine+½O₂−>Taurine (4)

L-Cysteic acid−>Taurine+CO₂ (5)
(1): In a first step, L-cysteine is oxidized by the enzyme cysteine dioxygenase (CDO, EC 1.13.11.20) to L-cysteine sulfinic acid (3-sulfinoalanine, CAS number 207121-48-0)
(3) and (5): Cysteine sulfinate decarboxylase (CSAD, EC 4.1.1.29) decarboxylates L-cysteine sulfinic acid to hypotaurine (2-aminoethanesulfinic acid, CAS number 300-84-5) and can decarboxylate L-cysteic acid to taurine in a similar reaction.
(2) and (4): The oxidation of cysteine sulfinic acid to cysteic acid and the oxidation of hypotaurine to taurine have yet to be unambiguously clarified.

A disadvantage of the known biotechnological processes for the production of taurine is that hypotaurine is generally the main product. If taurine is to be the product, chemical processes for the oxidation of hypotaurine must be employed.

Honjoh et al. (2010), Amino Acids 38: 1173-1183 describe a genetically engineered yeast strain heterologously expressing the CDO gene and CSAD gene from carp (Cyprinus carpio). The main product was hypotaurine, alongside a smaller proportion of taurine. Both products accumulated intracellularly. Analysis of the products therefore necessitated cell disruption. In cell extracts hypotaurine could for analytical purposes be chemically oxidized to taurine by treatment with H₂O₂. No process has been described that details how hypotaurine can be converted into taurine in a non-chemical step for further use.

WO 17/213142 A1 (Ajinomoto) describes a taurine-producing strain obtained by heterologous expression of a cysteine dioxygenase and an L-cysteine sulfinic acid decarboxylase in an originally cysteine-producing strain. The main product was hypotaurine with a maximum yield of 450 μM, which could be converted into taurine only through subsequent chemical treatment with alkali and only in low yields.

US 2019-0062757 A1 (KnipBio) describes heterologous production strains for the production of taurine or precursor substances thereof wherein the disclosed production strains mostly have a nonuniform product profile and achieve the highest yields for hypotaurine. The yields achieved were moreover very low, at max. 419 ng/ml hypotaurine, without any detectable taurine.

In addition to the low yields, the biotechnological approaches for the production of taurine disclosed in the prior art have the disadvantage of an inconsistent range of products, hypotaurine occurring as the main product and taurine only as a by-product.

An approach to the enzymatic oxidation of hypotaurine is disclosed by Veeravalli et al. (2020), bioRxiv Preprint Server doi: https://doi.org/10.1101/750273. They describe the mammalian flavin-dependent monooxygenase 1 (FMO1) as an enzyme for the conversion of hypotaurine into taurine. The oxidation proceeds according to equation (6):

Hypotaurine+NAD(P)H+O₂−>Taurine+NAD(P)+H₂O

It is not known whether the mammalian enzyme FMO1 for the conversion of hypotaurine into taurine according to equation (6) is also suitable for oxidizing other sulfinic acids of formula H₂N—CH(R)—CH₂—SO₂H.

For the oxidation of hypotaurine, FMO1 uses the cofactors NADH or NADPH in stoichiometric amounts. The use of these commercially costly cofactors makes technical use uneconomical. Furthermore, no production process suitable for industrial production is known for the FMO1 identified in mammals. There is thus no economic basis for enzymatic oxidation using the FMO1 enzyme.

The object of the present invention was therefore to provide an economically favorable biotechnological process for the oxidation of sulfinic acids to sulfonic acids, especially of L-cysteine sulfinic acid to L-cysteic acid and of hypotaurine to taurine.

The object was achieved by a process for the enzymatic oxidation of sulfinic acids of formula H₂N—CH(R)—CH₂—SO₂H to sulfonic acids of formula H₂N—CH(R)—CH₂—SO₃H with an enzyme selected from the class of H₂O₂-generating oxidases in the presence of the substrate of said enzyme. Preferably, the process is characterized in that the sulfinic acid is aminoalkyl sulfinic acid, more preferably 2-aminoalkyl sulfinic acid. Preferably, the process is characterized in that the sulfonic acid is aminoalkyl sulfonic acid, more preferably 2-aminoalkyl sulfonic acid. More preferably, the sulfinic acid is aminoalkyl sulfinic acid and the sulfonic acid aminoalkyl sulfonic acid

The radical R may be any radical and is preferably hydrogen, an organic, linear, branched, cyclic, saturated or unsaturated, aromatic or heteroaromatic radical with or without substituents. This means that the radicals R may be substituted or unsubstituted. Preferred substituents are —CN, —NCO, —NR₂, —COOH, —COOR, —halogen, -(meth)acryloyl, -epoxy, —SH, —OH, —CONR₂, —O—R, —CO—R, —COO—R, —OCO—R, or —OCOO—R, —S—R, —NR—, —N═R, —N═N—R, or —P═R. Preference is given to using saturated or unsaturated radicals with C₁-C₄ alkyl, more preferably C₁-C₄ alkyl, vinyl, in particular methyl or ethyl, especially methyl. R is preferably selected from R═H or R═CO₂H, i.e. it is preferable that the sulfinic acid is hypotaurine or cysteine sulfinic acid. Particular preference is given to R═H.

The invention thus relates also to the enzymatic oxidation of L-cysteine sulfinic acid to L-cysteic acid and of hypotaurine to taurine as a process step in a biotechnological production of taurine.

Substrate of the enzyme selected from the class of H₂O₂-generating oxidases is understood as meaning a substance oxidizable by the oxidase. The oxidase reacts with the oxidizable substrate thereof according to formula (7).

Substrate+O₂−>Substrate_{(OX)}+H₂O₂ (7)

The overall reaction catalyzed by the H₂O₂-generating oxidase takes place according to equation (8):

Substrate+O₂+H₂N—CH(R)—CH₂—SO₂H−> (8)

Substrate_{(OX)}+H₂O₂+H₂N—CH(R)—CH₂—SO₂H−>

Substrate_{(OX)}+H₂O+H₂N—CH(R)—CH₂—SO₃H

Suitable H₂O₂-generating oxidases can be found in the “KEGG enzyme” database under the search term “oxidase” as a subset of the enzymes listed there. However, of the multitude of H₂O₂-generating oxidases, the only ones potentially utilizable industrially are those oxidases that use an inexpensive substrate to generate H₂O₂ through the oxidation of the substrate according to equation (7).

Examples of preferred suitable H₂O₂-generating oxidases and not limited thereto are glucose oxidase (EC 1.1.3.4), hexose oxidase (EC 1.1.3.5), alcohol oxidase (EC 1.1.3.13), secondary alcohol oxidase (EC 1.1.3.18), pyranose oxidase (EC 1.1.3.10), L-lactate oxidase (EC 1.1.3.2), aryl-alcohol oxidase (EC 1.1.3.7), galactose oxidase (EC 1.1.3.9), L-sorbose oxidase (EC 1.1.3.11), aldehyde oxidase (EC 1.2.3.1), pyruvate oxidase (EC 1.2.3.3 or EC 1.2.3.6), oxalate oxidase (EC 1.2.3.4), glyoxylate oxidase (EC 1.2.3.5), L-amino acid oxidase (EC 1.4.3.2), D-amino acid oxidase (EC 1.4.3.3 or EC 1.4.3.1), sulfite oxidase (EC 1.8.3.1), and thiol oxidase (EC 1.8.3.2).

Particularly preferred H₂O₂-generating oxidases are glucose oxidase (EC 1.1.3.4), hexose oxidase (EC 1.1.3.5), alcohol oxidase (EC 1.1.3.13), secondary alcohol oxidase (EC 1.1.3.18), pyranose oxidase (EC 1.1.3.10), and L-lactate oxidase (EC 1.1.3.2).

Preferably, the process according to the invention is accordingly characterized in that the combination of H₂O₂-generating oxidase and the oxidizable substrate thereof is selected from glucose oxidase/glucose, alcohol oxidase/methanol, alcohol oxidase/ethanol, secondary alcohol oxidase/isopropanol and L-lactate oxidase/lactate.

In a preferred embodiment, the process is characterized in that the H₂O₂-generating oxidase is alcohol oxidase and that the substrate present is a primary alcohol, more preferably methanol. That is to say, the H₂O₂-generating oxidase is an enzyme of class EC 1.1.3.13 of the KEGG database designated alcohol oxidases.

Alcohols are compounds of general formula R—OH that have one or more hydroxy groups and do not contain any other functional group having higher priority. Alcohols are differentiated according to the number of carbon and hydrogen atoms on the carbon atom of the functional group to which the hydroxyl group is also attached. In the case of primary alcohols, in addition to a carbon atom two hydrogen atoms are attached to said carbon atom, giving a general formula of RCH₂OH. In addition, a primary alcohol is into an aldehyde by oxidation. For example, ethanol becomes the aldehyde ethanal through the elimination of 2 hydrogen atoms.

Preferred primary alcohols include methanol and ethanol, more preferably the primary alcohol is methanol.

R in the alcohols is an alkyl, alkenyl, or alkynyl radical, but not an aryl radical, acyl radical or a heteroatom.

In an alternatively preferred embodiment, the process is characterized in that the H₂O₂-generating oxidase is glucose oxidase and that the substrate present is glucose. That is to say, the H₂O₂-generating oxidase is an enzyme of class EC 1.1.3.4 of the KEGG database designated glucose oxidase.

A prerequisite for the industrial implementation of the process is the availability of the H₂O₂-generating oxidase and an inexpensive oxidizable substrate.

H₂O₂-generating oxidases can be produced for example by culturing a suitable production strain that produces the oxidase concerned (homologous production) naturally or as a result of expression of a suitable recombinant gene construct in a host organism (heterologous production). In addition, various H₂O₂-generating oxidases are commercially available, which can have a positive influence on the economics of the process. Examples of commercially available oxidases are alcohol oxidase AOX from the yeast Pichia pastoris (available for example from Sigma-Aldrich) or glucose oxidase GOX from the fungus Aspergillus niger (from homologous or heterologous production, available for example from Sigma-Aldrich). Another example of a commercially available glucose oxidase is the enzyme marketed under the trade name Gluzyme® (Novozymes) for uses e.g. in the baking industry. It is preferable that the H₂O₂-generating oxidase is produced by fermentation.

In addition, the inexpensive availability of the substrate of the H₂O₂-generating oxidase is critical to the economic viability of the process. In the case of AOX alcohol oxidase, the substrate is a primary alcohol, preferably selected from methanol or ethanol. The AOX enzyme generates H₂O₂ with methanol according to reaction (9):

Methanol+O₂−>Formaldehyde and H₂O₂ (9)

In the case of glucose oxidase GOX, the substrate is D-glucose. The GOX enzyme generates H₂O₂ with glucose according to reaction (10):

D-Glucose+O₂−>D-Glucono-1,5-lactone+H₂O₂ (10)

A process is for the purposes of the invention defined as a multistage sequence of work steps where, in one or more successive reaction batches, a reactant (starting material) is in a predefined sequence converted into the product via the intermediates determined by the reaction conditions.

A biotechnological process is for the purposes of the invention defined as the use of enzymes, cells or whole organisms in industrial applications for the production of chemical compounds, such as the production of taurine from hypotaurine using a glucose oxidase. By contrast with biotechnological processes, processes are that are characterized by chemical process steps.

A batch or reaction batch is defined as a mixture of reactant (starting material), enzyme, and optionally other reactants, in which the reactant is converted into a product under defined conditions. A batch or reaction batch of the present invention comprises at least one sulfinic acid of formula H₂N—CH(R)—CH₂—SO₂H, at least one enzyme selected from the class of H₂O₂-generating oxidases, the substrate oxidizable by the oxidase, and atmospheric oxygen O₂.

Biotransformation is defined as the conversion of a reactant into the product under enzymatic catalysis. The process according to the invention is a biotransformation.

The yield of the reaction can be expressed as a volume yield in terms of absolute amount of product per unit volume (mM or g/L) or as a relative yield of product as a percentage of reactant used (taking into account the molecular weights of the reactant and of the product), also referred to as percent yield. In the context of the invention, the molar yield, expressed in percent, refers to the total molar amount of sulfonic acid of formula H₂N—CH(R)—CH₂—SO₃H after enzymatic oxidation by an H₂O₂-generating oxidase in the presence of substrate thereof in relation to the sum total of the molar amount of sulfinic acid of formula H₂N—CH(R)—CH₂—SOand sulfonic acid of formula H₂N—CH(R)—CH₂—SO₃H used in this reaction.

Fermentation is a process step for the production of cell cultures in which a microbial production strain is made to grow under defined conditions of culture medium, temperature, pH, oxygen supply, and mixing of the medium. Depending on the configuration of the production strain, the aim of fermentation is to produce a protein/enzyme and/or a metabolite, in each case in the highest possible yield, for further use in a process.

The enzyme activity is expressed in U/ml, 1 U/ml being defined as the conversion of 1 μmol of substrate/min in 1 ml of test batch under test conditions.

Open reading frame (ORF, synonymous with cds or coding sequence) refers to a region of DNA or RNA that begins with a start codon and ends with a stop codon and encodes the amino acid sequence of a protein. The ORF is also referred to as the coding region or structural gene.

Gene or expression unit refers to the section of DNA that contains all the basic information for producing a biologically active RNA. A gene contains the section of DNA from which a single-stranded RNA copy is produced by transcription and also the expression signals involved in the regulation of this copying process. The expression signals include for example at least one promoter, a transcription start, a translation start, and a ribosome binding site. A terminator and one or more operators are additional possible expression signals.

An mRNA, also known as messenger RNA, is a single-stranded ribonucleic acid (RNA) that carries the genetic information for the synthesis of a protein. An mRNA provides the assembly instructions for a particular protein in a cell. The mRNA molecule conveys the requisite message for protein synthesis from the genetic information (DNA) to the ribosomes responsible for protein synthesis. In a cell it is formed as a transcript of a section of DNA corresponding to a gene. The genetic information stored in the DNA is unchanged by this process.

Genes of eukaryotic organisms are predominantly what are known as mosaic genes and, unlike prokaryotic genes, also contain non-coding sections known as introns (intragenic regions). Coding sequences, which are known as exons (pressed regions)), are sections of DNA of a eukaryotic gene that, after being transcribed into RNA, are translated by the ribosomes into the amino acid sequence of a protein. After transcription of DNA into RNA, the introns are spliced from the primary transcript. The protein-coding RNA freed of introns is termed messenger RNA (mRNA), or “mature” mRNA. This undergoes further modifications such as capping and polyadenylation. The coding region of the mature mRNA is then translated into the protein sequence. If a eukaryotic gene containing an exon/intron structure is to be expressed in prokaryotic organisms, it is necessary to back-translate the protein sequence or coding region of the mature mRNA into intron-free DNA, since no processing of the exon/intron structure takes place in prokaryotes. When referring in the context of this invention to gene sequences derived from the protein sequence or from mRNA, what is meant is precisely this process of back-translation. It is preferable that sequence optimization, i.e. adaptation to the codon usage of the corresponding prokaryote (codon optimization), takes place concomitantly with the back-translation of the mRNA sequence into a DNA sequence.

An operon is defined as a higher-level expression unit in which a multiplicity of genes is transcribed under the control of a single promoter, but wherein each is translated from its own ribosome binding site.

A gene construct is in the context of the invention a circular DNA molecule (plasmid, vector) in which at least one gene is linked to further genetic elements (for example promoter, ribosome binding site (RBS), terminator, selection marker, origin of replication). The genetic elements of the gene construct give rise to the extrachromosomal inheritance thereof during cell growth and to the production of the protein encoded by the gene.

As disclosed in examples 1 and 2 of the present invention, H₂O₂-generating oxidases in combination with the oxidizable substrate thereof are suitable for oxidizing sulfinic acids such as L-cysteine sulfinic acid and hypotaurine to the corresponding sulfonic acids L-cysteic acid and taurine respectively. This observation was novel and surprising. According to the prior art, albeit investigated largely only on an analytical scale (see for example Chauvin and Pratt, Angew. Chem. Int. Ed. (2017) 56: 6255-6259), H₂O₂ is in principle suitable for the oxidation of sulfinic acids to sulfonic acids. However, for a quantitative reaction H₂O₂ must according to equation (8) be used in at least stoichiometric amounts, which means that production of relatively large amounts of a sulfonic acid requires correspondingly large amounts of H₂O₂. Consequently, although H₂O₂-generating oxidases have been known for a long time, it has not been possible for those skilled in the art to foresee whether the amount of H₂O₂ arising from the alcohol oxidase reaction or the glucose oxidase reaction is sufficient to oxidize relatively large amounts of sulfinic acids. The process according to the invention thus has the advantage not only of being a biotechnological process, but also of being implementable on an industrial scale and of being an economically favorable process, since it does not require costly cofactors.

As disclosed for the first time in example 3 and not expected from the prior art, the use of glucose oxidase/glucose surprisingly achieved the almost quantitative oxidation of, for example, hypotaurine to taurine in a concentration relevant for chemical synthesis, viz. 20 g/L. The invention thus provides a biotechnological process that is suitable for the ever increasing demand for the sustainable production of sulfonic acids such as taurine for use in the foodstuffs, animal feed or pharmaceutical sectors, while avoiding chemical synthesis steps.

Preferably, the process is characterized in that the sulfinic acid is 2-aminoethanesulfinic acid (hypotaurine) and that the sulfonic acid formed is 2-aminoethanesulfonic acid (taurine).

The enzymatic process according to the invention for the oxidation of a sulfinic acid to the sulfonic acid is preferably carried out at a temperature of from 15° C. to 80° C., more preferably from 20° C. to 60° C., and especially preferably from 25° C. to 50° C.

The pH at which the process according to the invention can be carried out depends on the enzymatic properties of the H₂O₂-generating oxidase. As described in the examples, the reaction of the alcohol oxidase was carried out at pH 7.5 and that of the glucose oxidase at pH 5.5. The pH range in which the reaction is preferably carried out is pH 3.0 to pH 8.5, more preferably pH 4.0 to pH 8.0, and especially preferably pH 4.5 to pH 7.5.

The process according to the invention is carried out with the supply of atmospheric oxygen, either by passive input, as occurs by mixing the reaction batch, for example on an incubation shaker, or by active input, as occurs by passage of compressed air.

Alongside parameters such as temperature, pH, and oxygen input, the dosage of the H₂O₂-generating oxidase in the process according to the invention determines the conversion of the reaction and is chosen such that the reaction takes place with the lowest possible dosage of H₂O₂-generating oxidase in the shortest possible time. The dosage of the H₂O₂-generating oxidase, for example glucose oxidase, is preferably 1 U/ml to 200 U/ml, more preferably 2 U/ml to 150 U/ml, and especially preferably 4 U/ml to 100 U/ml.

The substrate oxidizable by the H₂O₂-generating oxidase is in the process according to the invention preferably used in an at least equimolar amount based on the molar content of the sulfinic acid (molar ratio of sulfinic acid to oxidizable substrate preferably at least 1:1). Particular preference is given to a molar ratio of sulfinic acid to oxidizable substrate of at least 1:2 and especially preferably of at least 1:5.

The reaction time depends on the dosage of the H₂O₂-generating oxidase and is preferably not more than 72 h, more preferably not more than 48 h, and especially preferably not more than 24 h.

The solvent used for the reaction is preferably water.

The concentration of the sulfinic acid in the batch is preferably at least 1 g/l, more preferably at least 10 g/l, and especially preferably at least 20 g/l.

The molar conversion of sulfinic acid to sulfonic acid in the biotransformation according to the invention is preferably at least 60%, more preferably at least 80%, and especially preferably at least 90%.

The process according to the invention for the oxidation of sulfinic acids can be operated in a discontinuous or continuous manner. In discontinuous operation (batch operation), all reactants are added to the batch in the course of the reaction and the batch is worked up after the reaction has ended.

In continuous operation, the oxidase enzyme is initially charged in the form of a stationary phase, for example in a membrane reactor or immobilized on a support, and the substrates comprising a sulfinic acid and a substrate oxidizable by the H₂O₂-generating oxidase as mobile phase are brought into contact with the stationary phase. The contact time of the mobile phase with the stationary phase is preferably set such that the sulfinic acid is able to react to form the sulfonic acid product.

Preference is given to discontinuous (batch) operation.

The sulfonic acid from the enzymatic oxidation according to the invention can either be used further directly without further workup steps or it can be enriched or purified by means of known methods. The degree of enrichment here depends on the further use.

In a preferred embodiment, the process is characterized in that the sulfonic acid, more preferably taurine, is isolated from the reaction batch.

Methods for isolating sulfonic acids, especially taurine, are known to those skilled in the art from processes for the isolation of, for example, amino acids. Examples include filtration, centrifugation, extraction, adsorption, ion-exchange chromatography, precipitation, crystallization.

The process according to the invention discloses a novel and unexpected route for the enzymatic oxidation of sulfinic acids to the corresponding sulfonic acids and is particularly suitable for the biotechnological production of L-cysteic acid and taurine.

In the process according to the invention, the progress of the reaction is monitored by determining the content of sulfinic acid (reactant) and sulfonic acid (product) at the start, at various times during the reaction, and at the end of the reaction. The content in a solution or cell culture of sulfinic acid of formula H₂N—CH(R)—CH₂—SO₂H, for example the hypotaurine content, or the content of sulfonic acids of formula H₂N—CH(R)—CH₂—SO₃H, for example the taurine content, can be determined as follows: The content can be quantified directly from an aliquot of the batch, provided the concentration of the sulfinic acid reactant at the start of the reaction is at least 0.1 g/L, corresponding to an equivalent concentration of the sulfonic acid product at the end of the reaction with complete conversion, determined for example by HPLC. This is done by taking a 1 ml aliquot of the batch, incubating this at 80° C. for 5 min, then removing all solid constituents, for example by centrifuging at maximum speed in a benchtop centrifuge for 5 minutes, and quantifying the supernatant by HPLC calibrated for the sulfinic acid or sulfonic acid concerned, as described for example in example 1 for hypotaurine and taurine. If the concentration of the sulfinic acid reactant at the start of the reaction is less than 0.1 g/L, the sample can be preconcentrated to increase the measurement accuracy for both the sulfinic acid reactant and the sulfonic acid product, for example by evaporating the sample and redissolving it in an appropriate volume of H₂O (for example 10% of the sample volume, corresponding to a 10×concentration).

If a culture broth containing sulfinic acid is used in the process according to the invention, it can be used in the quantification as is. Alternatively, it is also possible to first remove the cells from the culture broth, for example by centrifugation or filtration, and to use the resulting sulfinic acid-containing cell culture supernatant in the process according to the invention. It is also possible to isolate the sulfinic acid from the cell culture supernatant by means of methods known per se and to use the purified sulfinic acid in the process according to the invention.

The following biotransformation assay can be used for demonstration of the process according to the invention and to determine the conversion: The sulfinic acid-containing solution or culture, for example a hypotaurine-containing culture broth, preferably fermenter broth, or the corresponding cell culture supernatant, the purified or commercially available sulfinic acid, such as hypotaurine, is used to produce a biotransformation batch. The biotransformation batch on a laboratory scale has a batch volume of 10 ml and contains at least 0.1 g/L of sulfinic acid (as described above from the culture/culture broth/fermenter broth, from the centrifuged culture supernatant or as a purified substance and quantified accordingly or as a commercial product dosed accordingly). The batch volume can also be higher depending on the production scale and is at least 1 L on a preparative scale, for example. The H₂O₂-generating oxidase, preferably AOX or GOX, and the substrate oxidizable by this oxidase are added such that the dosage of the enzyme activity is at least 1 U/ml. When AOX is used, at least 1% (v/v) of methanol, i.e. at least 0.1 ml per 10 ml of reaction batch, is added as substrate of the AOX enzyme. As substrate of the GOX enzyme, glucose is added to the reaction batch in a dosage of at least 10 g/L. The biotransformation batch is supplied with atmospheric oxygen, either passively through mixing, for example on an incubation shaker or with a stirrer, in each case with admission of air, or actively by introducing compressed air or pure oxygen with or without mixing. The pH of the reaction is 7.5 when AOX is used. The pH of the reaction is 5.5 when GOX is used. The pH of the batch can be kept constant for example by equipping it with a pH electrode connected to a pH control unit that meters in a correction agent (alkali or acid) from a burette (the “pH-stat” method). The reaction temperature is 25° C. (AOX) or 30° C. (GOX). The progress of the reaction can be monitored through the consumption of the reactant hypotaurine and the formation of the product taurine, it being possible to determine hypotaurine and taurine quantitatively, for example by HPLC. The end time of the reaction is chosen according to the progress of the reaction. The chosen end of the reaction is preferably when the molar yield of sulfonic acid of formula H₂N—CH(R)—CH₂—SO₃H, for example taurine, is at least 60%, more preferably at least 80%, and especially preferably at least 90%. The sulfonic acids of formula H₂N—CH(R)—CH₂—SO₃H formed in the reaction batch is then available for further use.

Sulfinic acids can originate from chemical or fermentative production, it being preferable that the sulfinic acid used in the process according to the invention originates from fermentative production. More preferably, the process is characterized in that the sulfinic acid is hypotaurine and that this originates from fermentative production. When the sulfinic acids of formula H₂N—CH(R)—CH₂—SO₂H, for example hypotaurine, originates from fermentative production and a sulfonic acid of formula H₂N—CH(R)—CH₂—SO₃H, for example taurine, is formed therefrom in the process according to the invention, it is a major advantage that the present invention provides a biotechnological process for the production of sulfonic acids of formula H₂N—CH(R)—CH₂—SO₃H, for example taurine. This process does not involve any chemical reaction steps.

Suitable for the production of sulfinic acids of formula H₂N—CH(R)—CH₂—SO₂H, for example hypotaurine, are bacterial strains (for example E. coli, Corynebacterium glutamicum, Pantoea ananatis, Bacillus subtilis), algae (for example Chlamydomonas reinhardtii), yeasts (for example Saccharomyces cerevisiae, Yarrowia lipolytica) or fungi (for example Aspergillus niger).

When the sulfinic acid of formula H₂N—CH(R)—CH₂—SO₂H is hypotaurine, a microorganism strain suitable for hypotaurine production is used. A microorganism strain suitable for hypotaurine production is characterized in that it contains a metabolic pathway that leads to hypotaurine according to one of the metabolic pathways specified in the KEGG Pathway Database “Taurine and hypotaurine metabolism”. Since the biosynthesis of hypotaurine according to equations (1) and (3) starts from L-cysteine, preference is given to a microorganism strain that is also suitable for cysteine production. As described for example by Wada and Takagi, Appl. Microbiol. Biotechnol. (2006) 73: 48-54, cysteine production in a wild-type microorganism is closely regulated, which means that a microorganism strain that has a regulated cysteine biosynthetic pathway, as documented in the prior art, is not suitable for the production of commercially viable amounts of hypotaurine. Preference is therefore given to a microorganism strain suitable for hypotaurine production that has a deregulated cysteine biosynthetic pathway.

A microorganism strain that has a deregulated cysteine biosynthetic pathway and is thus suitable for cysteine production is characterized in that it exhibits at least one of the following modifications:
a) The microorganism strain has the characteristic feature of a modified serA gene encoding a 3-phosphoglycerate dehydrogenase (SerA) in which feedback inhibition by L-serine is reduced by a factor of at least two relative to the corresponding wild-type enzyme (as described for example in EP 1 950 287 B1), where the SerA enzyme activity can be determined for example photometrically through the oxidation of NADH dependent on the SerA substrate 3-phosphohydroxypyruvate, as described for example by McKitrick and Pizer, J. Bacteriol. (1980) 141: 235-245.
In particularly preferred variants of the 3-phosphoglycerate dehydrogenase (serA), feedback inhibition by L-serine is reduced by a factor of at least 5 relative to the corresponding wild-type enzyme, especially preferably by a factor of at least 10 and, in a more preferred embodiment, by a factor of at least 50. and/or
b) The microorganism strain contains a modified cysE gene encoding a serine-O-acetyl-transferase (CysE) in which feedback inhibition by cysteine is reduced by a factor of at least two relative to the corresponding wild-type enzyme (as described for example in EP 0 858 510 B1 or Nakamori et al., Appl. Env. Microbiol. (1998) 64: 1607-1611), where the CysE enzyme activity can be determined for example photometrically through the consumption of the CysE substrate acetyl-CoA resulting from the reaction with L-serine to O-acetyl-L-serine, as described for example by Nakamori et al., Appl. Env. Microbiol. (1998) 64: 1607-1611). In particularly preferred variants of the serine O-acetyltransferase (cysE), feedback inhibition by cysteine is reduced by a factor of at least 5 relative to the corresponding wild-type enzyme, especially preferably by a factor of at least 10 and, in a more preferred embodiment, by a factor of at least 50.
and/or
c) In the microorganism strain, cysteine export out of the cell is as a result of overexpression of an efflux gene increased by a factor of at least two relative to the corresponding wild-type cell, where cysteine export can be determined by photometric measurement of the extracellular cysteine content according to Gaitonde, Biochem. J (1967) 104: 627-633 (comprising cysteine, cystine, and the adduct (R)-2-methylthiazolidine-2,4-dicarboxylic acid formed from cysteine and pyruvate), as described for example in EP 0 885 962 B1.
The overexpression of an efflux gene results in cysteine export out of the cell being increased relative to a wild-type cell preferably by a factor of at least 5, more preferably by a factor of at least 10, and especially preferably by a factor of at least 20.
The efflux gene preferably comes from the group consisting of ydeD (see EP 0 885 962 B1), yfiK (see EP 1 382 684 B1), cydDC (see WO 2004/113373 A1), bcr (see US 2005-221453 AA), and emrAB (see US 2005-221453 AA) from E. coli or the correspondingly homologous gene from a different microorganism.

The microorganism strain suitable for hypotaurine production is used for production of hypotaurine according to the prior art, preferably by fermentation. A hypotaurine-containing culture broth, preferably a fermenter broth, is formed. The content of hypotaurine and also taurine, if present as a by-product, can be quantified from the culture broth. This is done by taking a 1 ml aliquot of the culture broth having a cell density OD₆₀₀/ml of at least 1.0/ml, incubating this at 80° C. for 5 min, then removing all solid constituents, for example by centrifuging at maximum speed in a benchtop centrifuge for 5 minutes, and quantifying the supernatant by HPLC calibrated for hypotaurine and taurine, as described for example for hypotaurine and taurine in example 1.

Those skilled in the art can use isotope analysis to determine whether a substance such as hypotaurine that he/she wishes to use as a sulfinic acid in the process comes from chemical or fermentative production. An isotope analysis method capable of differentiating is described for example in Sieper et al., Rapid Commun. Mass Spectrom. (2006) 20: 2521-2527 and is based on determination of the isotope ratios for e.g. carbon or nitrogen, which vary according to whether a product comes from chemical (petroleum-based) or natural (plant-based) production. The process for producing hypotaurine described in example 5 counts as a natural (plant-based) production process, since the glucose used for culturing the production strain originated from plant-based production according to the prior art.

Homologous genes, proteins or homologous sequences are to be understood as meaning that the DNA sequences or amino acid sequences of said genes, sections of DNA or proteins are at least 70% identical, preferably at least 80% identical and more preferably at least 90% identical.

The degree of DNA identity is determined by the “nucleotide blast” program, which can be found at http://blast.ncbi.nlm.nih.gov/ and is based on the blastn algorithm. The default parameters served as the algorithm parameters for an alignment of two or more nucleotide sequences. The default general parameters are: Max target sequences=100; Short queries=“Automatically adjust parameters for short input sequences”; Expect Threshold=10; Word size=28; Automatically adjust parameters for short input sequences=0. The corresponding default scoring parameters are: Match/Mismatch Scores=1, −2; Gap Costs=Linear.

Protein sequences are compared using the “protein blast” program at http://blast.ncbi.nlm.nih.gov/. This program uses the blastp algorithm. The default parameters served as the algorithm parameters for an alignment of two or more protein sequences. The default general parameters are: Max target sequences=100; Short queries=“Automatically adjust parameters for short input sequences”; Expect Threshold=10; Word size=3; Automatically adjust parameters for short input sequences=0. The default scoring parameters are: Matrix=BLOSUM62; Gap Costs=Existence: 11 Extension: 1; Compositional adjustments=Conditional compositional score matrix adjustment.

In a preferred embodiment, the process is characterized in that the sulfinic acid is hypotaurine and that this hypotaurine originates from bacterial production, i.e. that it was produced using a bacterial production strain. The bacterial production strain is preferably a strain of the species Escherichia coli.

The preferred bacterial production strain also has the characteristic feature of being suitable for cysteine production. Suitable and preferred strains for cysteine production are the E. coli K12 W3110 x pCys and E. coli K12 W3110-ppsA-MHI x pCys strains disclosed in the examples. As disclosed in example 5 of the present invention, heterologous expression of a CDO gene encoding a cysteine dioxygenase and of a CSAD gene encoding a cysteine sulfinic acid decarboxylase in the strain W3110 x pCys-CDOrn-CSADhs derived from W3110 x pCys and in the strain W3110-ppsA-MHI x pCys-CDOrn-CSADhs derived from W3110-ppsA-MHI x pCys results in the production of hypotaurine and only little taurine.

A microorganism strain designated ppsA-MHI is characterized in that it lacks the coding sequence of the Wt-ppsA gene and that this has been replaced by the ppsA-MHI cds, SEQ ID NO: 9 encoding a protein having the amino acid sequence SEQ ID NO: 10.

ppsA refers to the gene encoding a protein having the enzyme activity of the enzyme class designated EC 2.7.9.2 in the KEGG database. The corresponding protein is also referred to as PpsA or as phosphoenolpyruvate synthase (PEP synthase) or else synonymously as phosphoenolpyruvate-H₂O dikinase. The enzyme class is defined as being able to produce pyruvate from phosphoenolpyruvate in a reversible reaction according to the formula:

Phosphoenolpyruvate+phosphate+AMP<−>Pyruvate+H₂O+ATP (11)

As disclosed in example 6, the hypotaurine from such batches can be completely converted into taurine in a hitherto unknown manner by using a glucose oxidase together with D-glucose. The process according to the invention thus permits the biotechnological production of taurine by combining the biosynthetic production of hypotaurine with the enzymatic oxidation thereof to taurine.

In a development and simplification of the process, it is also conceivable to express the H₂O₂-generating oxidase in the hypotaurine production strain. Genes for suitable H₂O₂-generating oxidases can be identified in databases such as NCBI (National Center for Biotechnology Information). An example of a heterologous glucose oxidase produced in E. coli is the GOX gene from Penicillium amagasakiense (Witt et al., App. Environ. Microbiol (1998) 64: 1405-1411).

Preference is given to a biotechnological process for the production of taurine in which, in a first step, hypotaurine is produced by culturing a production strain and, in a second step, the hypotaurine formed is without further processing oxidized enzymatically to taurine, the enzymatic oxidation of hypotaurine to taurine by glucose oxidase/D-glucose being particularly preferred. In the examples of the present invention, the biotechnological production of hypotaurine (example 5) and the enzymatic oxidation thereof to taurine (example 6) are disclosed.

In a preferred embodiment, the process is characterized in that the sulfinic acid is hypotaurine and that this hypotaurine originates from bacterial production, i.e. that it was produced using a bacterial production strain having a deregulated cysteine biosynthetic pathway. The microorganism strain having a deregulated cysteine biosynthetic pathway is as per the above definition.

More preferably, cysteine is produced first, which reacts further according to equation (1) to cysteine sulfinic acid (CDO reaction) through the CDO enzyme present in the production strain and according to equation (3) to hypotaurine (CSAD reaction) through the CSAD enzyme likewise present in the production strain.

Particularly preferably, the process is characterized in that the sulfinic acid is hypotaurine produced by means of a bacterial production strain and that the production strain is one of the strains E. coli K12 W3110 x pCYS-CDOrn-CSADhs or E. coli K12 W3110-ppsA-MHI x pCYS-CDOrn-CSADhs, more preferably the strain E. coli K12 W3110-ppsA-MHI x pCYS-CDOrn-CSADhs.

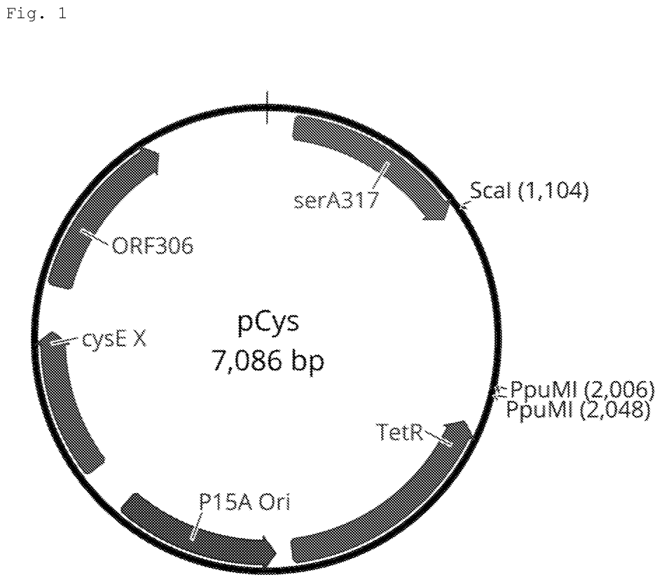

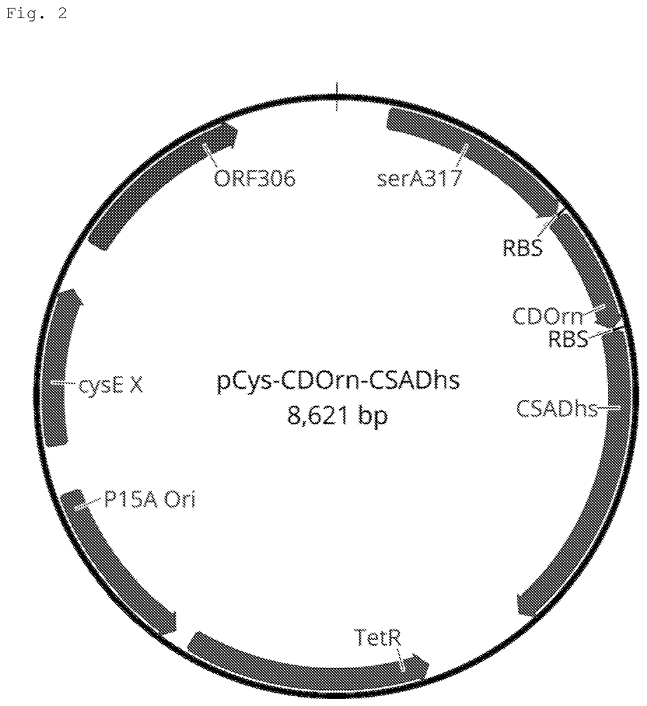

The invention thus also demonstrates production strains for the biotechnological production of hypotaurine. A particularly preferred starting strain is one of the strains E. coli K 12 W3110 x pCys E. coli K 12 W3110-ppsA-MHI x pCys suitable for cysteine production. As described in example 4 of the present invention, the vector pCys (FIG. 1) was extended with the genes for cysteine dioxygenase from Rattus norvegicus (CDorn), SEQ ID NO: 1, encoding a protein having the amino acid sequence SEQ ID NO: 2 and for L-cysteine sulfinic acid decarboxylase from homo sapiens (CSADhs), SEQ ID NO: 3, nt 1 to nt 1509, encoding a protein having the amino acid sequence SEQ ID NO: 4. This gave rise to the vector pCys-CDOrn-CSADhs suitable for hypotaurine production (FIG. 2).

The CDO gene used here is not confined to the species Rattus norvegicus. Any CDO gene for which the mRNA-derived gene product (protein) according to equation (1) is suitable for oxidizing L-cysteine to L-cysteine sulfinic acid is suitable. Such proteins can be found for example in the NCBI database by searching for CDO proteins in the “protein” sub-database using the search term “cysteine dioxygenase”. Other CDO proteins known from the prior art and not limited thereto are preferably the CDO protein from Homo sapiens (human), Cyprinus carpio (carp) or Synechococcus (algae) or a protein having an amino acid sequence 70% homologous thereto, more preferably a protein having an amino acid sequence 80% homologous thereto and, especially preferably, the amino acid sequence of the CDO protein has the sequence specified in SEQ ID NO: 2 and is encoded by the DNA sequence specified in SEQ ID NO: 1.

Likewise, the CSAD gene used is not confined to the species Homo sapiens. Any CSAD gene for which the mRNA-derived gene product (protein) according to equation (3) is suitable for decarboxylating L-cysteine sulfinic acid to hypotaurine is suitable. Such proteins can be found for example in the NCBI database by searching for CSAD proteins in the “protein” sub-database using the search term “cysteine sulfinic acid decarboxylase”. Other CSAD proteins known from the prior art and not limited thereto are preferably the CSAD protein from Rattus norvegicus (rat), Cyprinus carpio (carp), Synechococcus (algae) or E. coli or a protein having an amino acid sequence 70% homologous thereto, more preferably a protein having an amino acid sequence 80% homologous thereto and, especially preferably, the amino acid sequence of the CSAD protein has the sequence specified in SEQ ID NO: 4 and is encoded by the DNA sequence specified in SEQ ID NO: 3 from nt 1-1509.

A gene construct containing the CDO gene and CSAD gene, for example particularly preferably the construct pCys-CDOrn-CSADhs, can be produced according to the prior art using recombinant DNA techniques known to those skilled in the art, as described in detail by way of example in example 4.

This is done by cloning a CDO gene encoding the cysteine dioxygenase and a CSAD gene encoding a cysteine sulfinic acid decarboxylase into a vector suitable for this purpose. In principle, any vector with which the CDO gene and CSAD gene can be expressed in a production strain is suitable.

To produce a gene construct containing the CDO gene and CSAD gene, the CDO gene and CSAD gene can each be cloned into the vector, each with its own promoter and optionally a terminator, as separate expression units or else, in any desired sequence, as an operon under the control of a single promoter. Also possible is an operon structure in which the CDO gene and CSAD gene, each with its own ribosome binding site, are cloned after one of the genes already present in the vector and are expressed under the control of the promoter of the gene concerned. In addition, all other conceivable combinations of expression under their own promoters and in the form of an operon are possible for the CDO and the CSAD genes.

It is also conceivable for the CDO gene and CSAD gene to be cloned into a separate vector as separate expression units independently of the vector that ensures deregulated cysteine biosynthesis, for example pCys, or integrated into the genome of a host organism such as E. coli.

The preferred form for the expression of the CDO gene and CSAD gene is that of an artificial operon, in each case with its own ribosome binding site (RBS), but without its own promoter.

Particularly preferably, the vector is pCys-CDOrn-CSADhs in which the CDO gene and CSAD gene are cloned after the serA317 gene present in pCys, so that their expression takes place under the control of the serA317 promoter in accordance with the following sequence of expression units: serA promoter->(serA317-cds)->RBS-(CDOrn-cds)->RBS-(CSADhs-cds)-rrnB terminator. It is also possible for the expression units to be in a configuration in which the CSAD cds is arranged before the CDO cds.

pCys-CDOrn-CSADhs has the additional characteristic feature of ensuring deregulated cysteine biosynthesis by including the expression units for the feedback-resistant variant serA317 of 3-phosphoglycerate dehydrogenase (serA gene), the feedback-resistant variant CysEX of serine O-acetyl transferase (cysE gene), and the ydeD gene of a cysteine efflux protein. The invention relates to vectors in which at least one, preferably two, and more preferably three, of the genes serA317, cysEX and ydeD are present, it being possible for the genes concerned to be present in the vector in any desired selection and sequence.

When CDO and CSAD have been cloned into a vector, this is then transformed in a known manner into a suitable host strain. A suitable host strain is any microorganism that is accessible to recombinant DNA techniques and is suitable for the fermentative production of recombinant proteins. Preferred host strain is a strain of the species Escherichia coli, more preferably the strain E. coli K12 W3110 or E. coli K12 W3110-ppsA-MHI. The strains obtained from transformation, such as particularly preferably W3110 x pCys-CDOrn-CSADhs or W3110-ppsA-MHI x pCys-CDOrn-CSADhs, are suitable for the production of hypotaurine, as described in example 5.

The production of hypotaurine using particularly preferably one of the strains E. coli K12 W3110 x pCys-CDOrn-CSADhs or E. coli K12 W3110-ppsA-MHI x pCys-CDOrn-CSADhs can be accomplished by culturing in shake flasks (as described in example 5) or on a production scale by fermentation of the strain. In the process according to the invention, the hypotaurine formed is converted into taurine by enzymatic oxidation. This is done by incubating the hypotaurine-containing cell culture with an H₂O₂-generating oxidase in the presence of the substrate thereof, for example with a glucose oxidase in the presence of glucose (see example 6).

In a preferred embodiment, the process is characterized in that the sulfinic acid is cysteine sulfinic acid and that the sulfonic acid formed is cysteic acid.

Preferably, the process is characterized in that the molar yield of sulfonic acids of formula H₂N—CH(R)—CH₂—SO₃H based on the employed additive molar concentration of sulfinic acids of formula H₂N—CH(R)—CH₂—SO₂H and sulfonic acids of formula H₂N—CH(R)—CH₂—SO₃H is at least 60%, more preferably at least 80%, and especially at least 90%. The amount of sulfinic acid used is preferably at least 1 mM (109.2 mg/L), more preferably at least 10 mM (1.1 g/L), and especially preferably at least 100 mM (10.9 g/L).

To determine the value of the molar yield, the molar content of the sulfinic and sulfonic acids used in the enzymatic oxidation is determined at the start and at the end of the reaction by HPLC, as is known in the prior art. For example, the determination can be carried out as described in example 1, based on the molecular weights 109.2 g/mol for hypotaurine and 125.2 g/mol for taurine. The molar content of sulfonic acid in the batch at the end of the reaction in relation to the total molar content of sulfinic acid and sulfonic acid at the start of the reaction gives the molar yield of the sulfonic acid in percent.

The figures show the plasmids used in the examples.

FIG. 1: pCys.

FIG. 2: pCys-CDOrn-CSADhs.

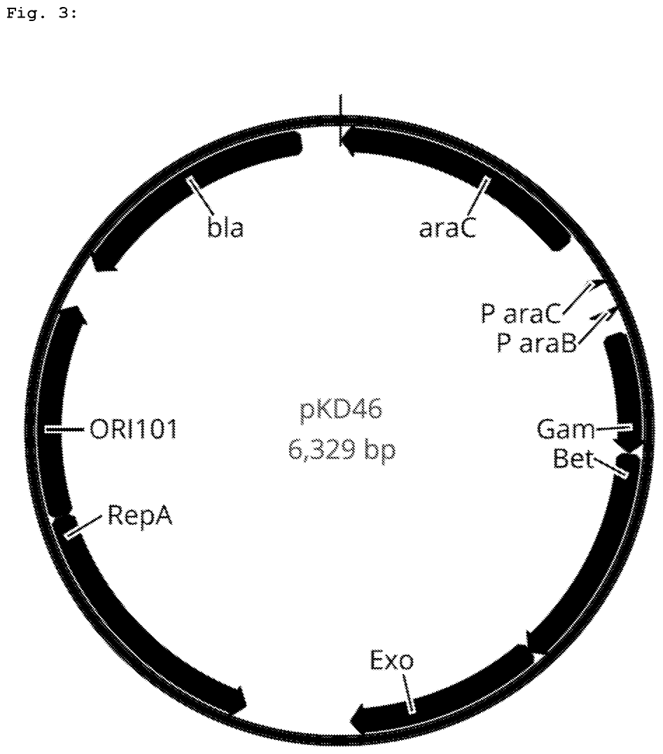

FIG. 3: pKD46.

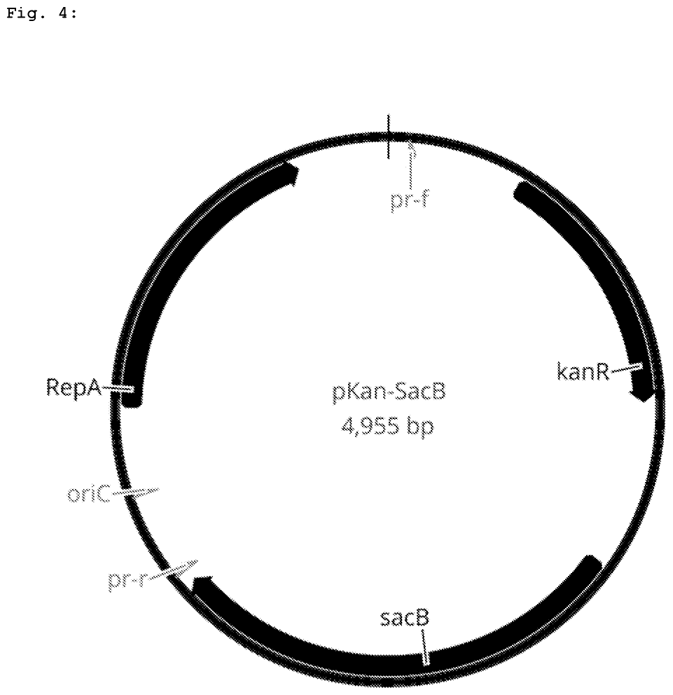

FIG. 4: pKan-SacB.

### ABBREVIATIONS USED IN THE FIGURES

bla: Gene conferring resistance to ampicillin (β-lactamase)

kanR: Gene conferring resistance to kanamycin

araC: araC gene (repressor gene)

P araC: Promoter of the araC gene

P araB: Promoter of the araB gene

Gam: Lambda phage Gam recombination gene

Bet: Lambda phage Bet recombination gene

Exo: Lambda phage Exo recombination gene

ORI101: Temperature-sensitive origin of replication

RepA: Gene for plasmid replication protein A

sacB: Levansucrase gene

pr-f: Binding site f for primer (forward)

pr-r: Binding site r for primer (reverse)

OriC: Origin of replication C

TetR: Gene conferring resistance to tetracycline

P15A ORI: Origin of replication

serA317: serA (3-phosphoglycerate dehydrogenase gene encoding amino acids 1 to 317) cds

cysE X: cysE (serine O-acetyltransferase gene, feedback resistant) cds

ORF306: ydeD (cysteine efflux gene) cds

ScaI: Cleavage site for the restriction enzyme ScaI

PpuMI: Cleavage site for the restriction enzyme PpuMI

CDOrn: CDO (cysteine dioxygenase) R. norvegicus cds

CSADhs: CSAD (cysteine sulfinic acid decarboxylase) H. sapiens cds

RBS: Ribosome binding site

The following examples serve to further elucidate the invention further without restricting it thereto.

### EXAMPLES

### Example 1

### Oxidation of Hypotaurine and Cysteine Sulfinic Acid with Alcohol Oxidase (AOX)

A) Oxidation of Cysteine Sulfinic Acid to Cysteic Acid with AOX:
The reaction was investigated in two parallel batches, i.e. with and without AOX. 12 mg of L-cysteine sulfinic acid monohydrate (Sigma-Aldrich) was weighed into each of two 100 ml conical flasks; this was dissolved in 9.9 ml of 100 mM Na phosphate pH 7.5 and 0.1 ml of methanol was added (end concentration 1% v/v). To start the reaction, 30 μl of a commercially available solution of AOX from Pichia pastoris (Sigma-Aldrich) in 100 mM Na phosphate pH 7.5 was added to one batch. In accordance with the manufacturer's information on the enzyme activity, the AOX activity in the batch was 5 U/ml. The second batch without AOX (comparison batch) was treated with 30 μl of 100 mM Na phosphate pH 7.5. The batches were shaken at 25° C. and 140 rpm (Infors incubator shaker). At the start and 5 h after the start of the reaction, 1 ml from each batch was taken, incubated at 80° C. for 5 min, and centrifuged at 13 000 rpm for 5 min (Heraeus™ Fresco™ 21 centrifuge) and the supernatants analyzed by HPLC. The content of L-cysteine sulfinic acid and L-cysteic acid in the batch with AOX and in the batch without AOX at the start (0 h) and after a reaction time of 5 h is summarized in Table 1.

TABLE 1
Time course of the oxidation of L-cysteine sulfinic acid to L-
cysteic acid by AOX in the presence of methanol

| | +5 U/ml AOX | | without AOX | |
|---|---|---|---|---|
| | L-Cysteine | | L-Cysteine | |
| | sulfinic | L-Cysteic | sulfinic | L-Cysteic |
| | acid | acid | acid | acid |
| Time | [mg/L] | [mg/L] | [mg/L] | [mg/L] |
| 0 h | 1171.5 | 0.0 | 1239.3 | 0.0 |
| 5 h | 13.4 | 1023.3 | 1244.3 | 0.0 |

B) Oxidation of Hypotaurine to Taurine with AOX:
The reaction was investigated in two parallel batches, i.e. with and without AOX. 12 mg of hypotaurine (Sigma-Aldrich) was weighed into each of two 100 ml conical flasks; this was dissolved in 9.9 ml of 100 mM Na phosphate pH 7.5 and 0.1 ml of methanol was added (end concentration 1% v/v). To start the reaction, 30 μl of a commercially available solution of AOX from Pichia pastoris (Sigma-Aldrich) in 100 mM Na phosphate pH 7.5 was added to one batch. In accordance with the manufacturer's information on the enzyme activity, the AOX activity in the batch was 5 U/ml. The batch without AOX (comparison batch) was treated with 30 μl of 100 mM Na phosphate pH 7.5. The batches were shaken at 25° C. and 140 rpm (Infors incubator shaker). At the start and 5 h after the start of the reaction, 1 ml from each batch was taken, incubated at 80° C. for 5 min, and centrifuged at 13 000 rpm for 5 min (Heraeus™ Fresco™ 21 centrifuge) and the supernatants analyzed by HPLC. The content of hypotaurine and taurine in the batch with alcohol oxidase (AOX) and in the batch without alcohol oxidase at the start (0 h) and after a reaction time of 5 h is summarized in Table 2.

TABLE 2
Time course of the oxidation of hypotaurine to
taurine by AOX in the presence of methanol

| | +5 U/ml AOX | | without AOX | |
|---|---|---|---|---|
| | Hypotaurine | Taurine | Hypotaurine | Taurine |
| Time | [mg/L] | [mg/L] | [mg/L] | [mg/L] |
| 0 h | 1030.2 | 0.0 | 1112.3 | 0.0 |
| 5 h | 0.0 | 1124.3 | 1169.3 | 0.0 |

### HPLC Analysis of L-Cysteine Sulfinic Acid, L-Cysteic Acid, Hypotaurine, and Taurine:

For quantitative determination of the compounds quantitatively analyzed in the examples, an HPLC method calibrated respectively for L-cysteine sulfinic acid, L-cysteic acid, hypotaurine, and taurine was employed; all reference substances used for calibration were commercially available (Sigma-Aldrich). An Agilent 1260 Infinity II HPLC system was used, which was equipped with a unit from the same manufacturer for pre-column derivatization with o-phthaldialdehyde (OPA derivatization) as is known from the analysis of amino acids. For detection of the OPA-derivatized products of L-cysteine sulfinic acid, L-cysteic acid, hypotaurine, and taurine, the HPLC system was equipped with a fluorescence detector. The detector was set to an excitation wavelength of 330 nm and an emission wavelength of 450 nm. Also used were an Accucore™ aQ column from Thermo Scientific™, length 100 mm, internal diameter 4.6 mm, particle size 2.6 μm, thermally equilibrated at 40° C. in a column oven.

Eluent A: 25 mM Na phosphate pH 6.0

### Eluent B: Methanol

The separation was carried out in gradient mode: 10% eluent B to 60% eluent B over min, followed by 60% eluent B to 100% eluent B over 2 min, followed by 100% eluent B for a further 2 min, at a flow rate of 0.5 ml/min. Retention time of L-cysteic acid: 3.2 min. Retention time of L-cysteine sulfinic acid: 4.1 min. Retention time of taurine: 14.8 min. Retention time of hypotaurine: 15.7 min.

### Example 2

### Oxidation of Hypotaurine and Cysteine Sulfinic Acid with Glucose Oxidase (GOX)

A) Oxidation of Cysteine Sulfinic Acid to Cysteic Acid with GOX:
The reaction was investigated in two parallel batches, i.e. with and without GOX. 12 mg of L-cysteine sulfinic acid monohydrate (Sigma-Aldrich) was weighed into each of two 100 ml conical flasks; this was dissolved in 9.5 ml of 100 mM Na acetate pH 5.5 and 0.5 ml of a 200 g/L glucose solution in the same buffer was added. To start the reaction, 50 μl of a commercially available solution of GOX from Aspergillus niger (Sigma-Aldrich) in 100 mM Na acetate pH 5.5 was added to one batch. In accordance with the manufacturer's information, the GOX activity in the batch was 5 U/ml. The batch without GOX (comparison batch) was treated with 50 μl of 100 mM Na acetate pH 5.5. The batches were shaken at 30° C. and 140 rpm (Infors incubator shaker). At the start and 5 h after the start of the reaction, 1 ml from each batch was taken, incubated at 80° C. for 5 min, and centrifuged at 13 000 rpm for 5 min (Heraeus™ Fresco™ 21 centrifuge) and the supernatants analyzed by HPLC as described above.
The content of L-cysteine sulfinic acid and L-cysteic acid in the batch with GOX and in the batch without GOX at the start (0 h) and after a reaction time of 5 h is summarized in Table 3.

TABLE 3
Time course of the oxidation of L-cysteine sulfinic acid to L-
cysteic acid by GOX in the presence of glucose

| | | | without GOX | |
|---|---|---|---|---|
| | +5 U/ml GOX | | L-Cysteine | |
| | L-Cysteine | | sulfinic | L-Cysteic |
| | sulfinic | L-Cysteic | acid | acid |
| Time | acid [mg/L] | acid [mg/L] | [mg/L] | [mg/L] |
| 0 h | 1322.7 | 0.0 | 1146.3 | 0.0 |
| 5 h | 3.0 | 1279.3 | 1171.3 | 0.0 |

B) Oxidation of Hypotaurine to Taurine with GOX:
The reaction was investigated in two parallel batches, i.e. with and without GOX. 12 mg of hypotaurine (Sigma-Aldrich) was weighed into each of two 100 ml conical flasks; this was dissolved in 9.5 ml of 100 mM Na acetate pH 5.5 and 0.5 ml of a 200 g/L glucose solution in the same buffer was added. To start the reaction, 50 μl of a commercially available solution of GOX from Aspergillus niger (Sigma-Aldrich) in 100 mM Na acetate pH 5.5 was added to one batch. In accordance with the manufacturer's information, the GOX activity in the batch was 5 U/ml. The batch without GOX (comparison batch) was treated with 50 μl of 100 mM Na acetate pH 5.5. The batches were shaken at 30° C. and 140 rpm (Infors incubator shaker). At the start and 5 h after the start of the reaction, 1 ml from each batch was taken, incubated at 80° C. for 5 min, and centrifuged at 13 000 rpm for 5 min (Heraeus™ Fresco™ 21 centrifuge) and the supernatants analyzed by HPLC as described above.
The content of hypotaurine and taurine in the batch with GOX and in the batch without GOX at the start (0 h) and after a reaction time of 5 h is summarized in Table 4.

TABLE 4
Time course of the oxidation of hypotaurine to
taurine by GOX in the presence of glucose

| | +5 U/ml GOX | | without GOX | |
|---|---|---|---|---|
| | Hypotaurine | Taurine | Hypotaurine | Taurine |
| Time | [mg/L] | [mg/L] | [mg/L] | [mg/L] |
| 0 h | 1238.3 | 0.0 | 1037.4 | 0.0 |
| 5 h | 0.0 | 1519.7 | 1103.3 | 0.0 |

### Example 3

### Preparative Oxidation of Hypotaurine to Taurine with GOX

The reaction was investigated in two parallel batches, i.e. with different dosing of the enzyme GOX, the concentration of the hypotaurine substrate undergoing oxidation being 20 g/L in each case.

In batch 1, 200 mg of hypotaurine (Sigma-Aldrich) was weighed into a 100 ml conical flask, dissolved in 6.95 ml of 100 mM Na acetate pH 5.5, and 3 ml of a 200 g/L glucose solution in the same buffer was added. To start the reaction, 50 μl of a commercially available solution of GOX from Aspergillus niger (Sigma-Aldrich) in 100 mM Na acetate pH 5.5 was added. In accordance with the manufacturer's information on the enzyme activity, the GOX activity in the batch was 5 U/ml. In batch 2, 200 mg of hypotaurine (Sigma-Aldrich) was weighed into a 100 ml conical flask, dissolved in 6.5 ml of 100 mM Na acetate pH 5.5, and 3 ml of a 200 g/L glucose solution in the same buffer was added. To start the reaction, 500 μl of a commercially available solution of GOX from Aspergillus niger (Sigma-Aldrich) in 100 mM Na acetate pH 5.5 was added. In accordance with the manufacturer's information on the enzyme activity, the GOX activity in the batch was 50 U/ml.

Batches 1 and 2 were shaken at 30° C. and 140 rpm (Infors incubator shaker). 3 h, 6 h, and 24 h after the start of the reaction, 1 ml aliquots of each batch were taken, incubated at 80° C. for 5 min, and centrifuged at 13 000 rpm for 5 min (Heraeus™ Fresco™ 21 centrifuge) and the supernatants analyzed by HPLC as described above. The course of the reactions over time is summarized in Table 5.

TABLE 5
Time course of the oxidation of hypotaurine to taurine
by GOX in the presence of glucose

| | +5 U/ml GOX | | +50 U/ml GOX | |
|---|---|---|---|---|
| | Hypotaurine | Taurine | Hypotaurine | Taurine |
| Time | [g/L] | [g/L] | [g/L] | [g/L] |
| 0 h | 20.0 | 0.0 | 20.0 | 0.0 |
| 3 h | 12.3 | 6.8 | 8.1 | 10.9 |
| 6 h | 7.6 | 12.2 | 3.0 | 18.7 |
| 24 h | 0.5 | 22.3 | 0.6 | 23.4 |

### Example 4

### Production of the Hypotaurine Production Strains E. coli K12 W3110 x pCys-CDOrn-CSADhs and E. coli K12 W3110-ppsA-MHI x pCys-CDOrn-CSADhs

### Cysteine Dioxygenase CDOrn:

CDOrn: The amino acid sequence of cysteine dioxygenase from Rattus norvegicus is disclosed in the NCBI (National Center for Biotechnology Information) database under the sequence ID: AAH70509.1. The amino acid sequence was used to derive a DNA sequence codon-optimized for expression in E. coli (publicly available Eurofins Genomics GENEius software), which was synthetically produced (Eurofins Genomics). This DNA sequence, designated CDOrn, is disclosed in SEQ ID NO: 1 and encodes a protein having the amino acid sequence from SEQ ID NO: 2.

### Cysteine Sulfinic Acid Decarboxylase CSADhs:

CSADhs: The amino acid sequence of cysteine sulfinic acid decarboxylase (CSADhs) from homo sapiens is disclosed in the NCBI (National Center for Biotechnology Information) database under the sequence ID: XP_016861786.1. The amino acid sequence was used to derive a DNA sequence codon-optimized for expression in E. coli (publicly available Eurofins Genomics GENEius software). This DNA sequence, designated CSADhs, is disclosed in SEQ ID NO: 3, nt 1 to 1509 and encodes a protein having the amino acid sequence from SEQ ID NO: 4. The DNA sequence of the E. coli rrnB terminator (SEQ ID NO: 3, nt 1510 to 1842) was coupled to nt 1509. The DNA sequence of the rrnB terminator is disclosed in Orosz et al., Eur. J. Biochem. (1991) 201: 653-659. The DNA disclosed in SEQ ID NO: 3, consisting of the CSADhs cds and the rrnB terminator, was produced synthetically (Eurofins Genomics) and given the designation CSADhs-rrnB.

Production of the Vector pCys-CDOrn-CSADhs:
Vector pCys (FIG. 1) refers to the plasmid pACYC184-cysEX-GAPDH-ORF306-serA317, a derivative of the plasmid pACYC184-cysEX-GAPDH-ORF306 disclosed in EP 0 885 962 B1. The plasmid pACYC184-cysEX-GAPDH-ORF306 contains not only the origin of replication and a tetracycline resistance gene (parent vector pACYC184), but also the cysEX allele, which encodes a serine O-acetyltransferase having reduced feedback inhibition by cysteine, and also the efflux gene ydeD (ORF306), the expression of which is controlled by the constitutive GAPDH promoter.
   To obtain pACYC184-cysEX-GAPDH-ORF306-serA317, the serA317 gene fragment encoding the N-terminal 317 amino acids of the SerA protein from E. coli and disclosed in Bell et al., Eur. J. Biochem. (2002) 269: 4176-4184 (referred to therein as “NSD:317”) was cloned in pACYC184-cysEX-GAPDH-ORF306 after the ydeD (ORF306) efflux gene. SerA317 encodes a serine feedback-resistant variant of 3-phosphoglycerate dehydrogenase. The expression of serA317 is controlled by the serA promoter.
   As a consequence of deregulated biosynthesis, E. coli cells transformed with pCys produce cysteine, the starting product for the derived products cysteine sulfinic acid and hypotaurine.
Vector pCys-CDOrn-CSADhs (FIG. 2):
   pCys was cut with Scal and PpuMI. The 6.1 kb vector fragment thereby released was isolated by preparative agarose gel electrophoresis (QIAquick® Gel Extraction Kit, Qiagen).
   The CDOrn DNA was amplified from the synthetic DNA SEQ ID NO: 1 (CDOrn) by PCR (“Phusion™ High Fidelity” DNA polymerase, Thermo Scientific™) using the primers cdorn-1f (SEQ ID NO: 5) and csadhs-2r (SEQ ID NO: 6) and isolated as a kb fragment.
   Primer cdorn-1f comprised, starting from the 5′ end, 28 nt overlapping with the 3′ end of the 6.1 kb pCys Scal/PpuMI vector fragment (nt 1 to 28 in SEQ ID NO: 5) obtained by Scal digestion, a ribosome binding site (RBS) (nt 31 to 36 in SEQ ID NO: 5), and the first 22 nt of the CDOrn cds (SEQ ID NO 5, nt 44 to 65).
   Primer csadhs-2r comprised, in reverse complement form, starting from the 5′ end, 22 nt overlapping with the start of the CSADhs cds (SEQ ID NO: 6, nt 1 to 22) followed by a ribosome binding site (SEQ ID NO: 6, nt 30 to 35) and the last 20 nt of the CDOrn cds (SEQ ID NO: 6, nt 38 to 57).
CSADhs-rrnB DNA: The DNA fragment was amplified from the synthetic DNA SEQ ID NO: 3 (CSADhs-rrnB) by PCR (“Phusion™ High Fidelity” DNA polymerase, Thermo Scientific™) using the primers csadhs-3f (SEQ ID NO: 7) and glf-2r (SEQ ID NO: 8) and isolated as a 1.8 kb fragment.
   Primer csadhs-3f comprised, starting from the 5′ end, 20 nt overlapping with the 3′ end of the CDOrn cds (nt 1 to 20 in SEQ ID NO: 7), a ribosome binding site (nt 23 to 28 in SEQ ID NO: 7), and the first 22 nt of the CSADhs cds (SEQ ID NO 7, nt 36 to 57).
   Primer glf-2r comprised, in reverse complement form, starting from the 5′ end, 33 nt overlapping with the 5′ end of the 6.1 kb pCys Scal/PpuMI vector fragment (nt 1 to 33 in SEQ ID NO: 8) obtained by PpuMI digestion, followed by the last 22 nt of the rrnB terminator (SEQ ID NO: 8, nt 34 to 55).
Vector pCys-CDOrn-CSADhs: The 6.1 kb pCys Scal/PpuMI vector fragment, the 0.6 kb CDOrn PCR product (CDOrn DNA), and the 1.8 kb CSADhs-rrnB PCR product (CSADhs-rrnB DNA) were ligated using the NEBuilder® cloning kit (NEB New England Biolabs) according to the manufacturer's instructions.
   E. coli NEB ® 10-beta cells (NEB New England Biolabs) were then transformed with the ligation mixture. Clones from the transformation were selected on LBtet. LBtet contained 10 g/L of tryptone (Gibco™), 5 g/L of yeast extract (BD Biosciences), 5 g/L of NaCl, 15 g/L of agar, and 15 mg/L of tetracycline (Sigma-Aldrich). A single clone from the transformation was analyzed by culturing in LBtet liquid medium (10 g/L of tryptone, 5 g/L of yeast extract, 5 g/L of NaCl, and 15 mg/L of tetracycline) and isolating the vector from the cell pellet from culturing. The correct 8.5 kb vector was designated pCys-CDOrn-CSADhs (FIG. 2).
Strain E. coli W3110:
   The strain used was Escherichia coli K12 W3110 (commercially available under the strain number DSM 5911 from the DSMZ: Deutsche Sammlung von Mikroorganismen and Zellkulturen GmbH [German Collection of Microorganisms and Cell Cultures]).
Strain E. coli W3110-ppsA-MHI:
   The strain used was E. coli K12 W3110-ppsA-MHI. E. coli K12 W3110-ppsA-MHI is characterized by the mutated ppsA gene ppsA-MHI (SEQ ID NO: 9) encoding a protein having the protein sequence from SEQ ID NO: 10 and the enzymatic activity of a PEP synthase (enzyme class designated EC 2.7.9.2 in the KEGG database). E. coli K12 W3110-ppsA-MHI was produced by using the combination known to those skilled in the art of Lambda-Red recombination and counter-selection screening for genetic modification (see for example Sun et al., Appl. Env. Microbiol. (2008) 74: 4241-4245). To replace the ppsA WT gene of E. coli K12 W3110 with ppsA-MHI, the following steps were carried out:
      1) E. coli K12 W3110 was transformed with the plasmid pKD46 (FIG. 3, disclosed in the “GenBank” gene database under access number AY048746.1) and the strain E. coli W3110 x pKD46 was isolated (ampicillin selection).
      2) From the plasmid pKan-sacB (FIG. 4), the 3.2 kb Kan-sacB cassette was isolated by PCR using the primers pps-9f (SEQ ID NO: 11, binds to the site designated “pr-f” in FIG. 4), and pps-10r (SEQ ID NO: 12, binds to the site designated “pr-r” in FIG. 4). The plasmid pKan-sacB contains expression cassettes both for the kanamycin (Kan) resistance gene and for the sacB gene encoding the enzyme levansucrase. The E. coli kanamycin resistance gene (Kan) encoding an aminoglycoside phosphotransferase is disclosed in the NCBI database under access number SH02_03400. The B. subtilis sacB gene is disclosed in the NCBI database under access number 936413. Primer pps-9f contains the first 30 nt of the ppsA WT gene (identical to SEQ ID NO: 9, nt 1 to 30) and, connected thereto, 20 nt of the site designated “pr-f” in FIG. 4. Primer pps-10r contains the last 30 nt of the ppsA WT gene, in reverse complement form (identical to SEQ ID NO: 9, nt 2350 to 2379) and, connected thereto, 20 nt of the site designated “pr-r” in FIG. 4.
   E. coli W3110 x pKD46 was transformed with the 3.2 kb Kan-sacB cassette and kanamycin-resistant clones were isolated.
      4) The clones were seeded onto LBSC plates (10 g/L of tryptone, 5 g/L of yeast extract, 7% sucrose, 1.5% agar, and 15 mg/L of kanamycin). Clones having an integrated sacB gene produced toxic levan from sucrose, which led to growth inhibition (sucrose-sensitive). A kanamycin-resistant and sucrose-sensitive clone was selected and designated W3110-ppsA::Kan-sacB x pKD46.
      5) W3110-ppsA::Kan-sacB x pKD46 was transformed with DNA of the ppsA-MHI gene (SEQ ID NO: 9, produced synthetically, from Eurofins Genomics) and clones selected on LBS plates (10 g/L of tryptone, 5 g/L of yeast extract, 7% sucrose, 1.5% agar) without kanamycin. Only clones that no longer contained an active sacB gene were able to grow on LBS plates. These clones were seeded onto LBkan plates (10 g/L of tryptone, 5 g/L of yeast extract, 5 g/L of NaCl, 1.5% agar, 15 mg/L of kanamycin) in order to select those clones that also no longer contained an active Kan gene and the growth of which was inhibited in the presence of kanamycin.
      6) A clone exhibiting positive growth in the presence of sucrose and negative growth in the presence of kanamycin was selected, the ppsA MHI gene was isolated by PCR from genomic DNA of the strain, and DNA sequencing (Eurofins Genomics) confirmed that the ppsA MHI gene having the DNA sequence disclosed in SEQ ID NO: 9 had been integrated, encoding a protein corresponding to the sequence from SEQ ID NO: 10. The strain, after removal of the plasmid pKD46 by incubation at 42° C., was given the designation E. coli W3110-ppsA-MHI.
Production strains: Plasmid DNA of the vector pCys-CDOrn-CSADhs was transformed into the strains E. coli K12 W3110 and E. coli K12 W3110-ppsA-MHI. For comparison, E. coli K12 W3110 and E. coli K12 W3110-ppsA-MHI were transformed with the vector pCys. Transformants were selected on LBtet. One clone was in each case isolated. The strains were given the designation E. coli K12 W3110 x pCys-CDOrn-CSADhs and E. coli K12 W3110 x pCys and, by analogy, E. coli K12 W3110-ppsA-MHI x pCys-CDOrn-CSADhs and E. coli K12 W3110-ppsA-MHI x pCys respectively. E. coli K12 W3110 x pCys-CDOrn-CSADhs and E. coli K12 W3110-ppsA-MHI x pCys-CDOrn-CSADhs were used as production strains for the production of hypotaurine.

### Example 5

### Production of Hypotaurine in Shake Flasks

A preculture in LBtet liquid medium was produced from each of the strains E. coli K12 W3110 x pCys-CDOrn-CSADhs, E. coli K12 W3110 x pCys, E. coli K12 W3110-ppsA-MHI x pCys-CDOrn-CSADhs, and E. coli K12 W3110-ppsA-MHI x pCys (cultured overnight at 37° C. and 120 rpm).

Main culture: 0.5 ml of the preculture was transferred to a 300 ml conical flask (baffled) with 30 ml of SM1-Ac medium, also containing 15 g/L of glucose, 2 g/L of Na₂S₂O₃·5H₂O, 0.1 g/L of L-isoleucine, 0.1 g/L of D,L-methionine, 0.1 g/L of L-threonine, 5 mg/L of vitamin B1, and 15 mg/L of tetracycline.

Composition of the SM1-Ac medium: 12 g/L of K₂HPO₄, 3 g/L of KH₂PO₄, 5 g/L of NH₄ acetate, 0.3 g/L of MgSO₄·7H₂O, 0.015 g/L of CaCl₂·2H₂O, 0.002 g/L of FeSO₄·7H₂O, 1 g/L of trisodium citrate dihydrate, 0.1 g/L of NaCl; 1 ml/L of trace element solution.

Composition of the trace element solution: 0.15 g/L of Na₂MoO₄·2H₂O, 2.5 g/L of H₃BO₃, 0.7 g/L of CoCl₂·.6H₂O, 0.25 g/L of CuSO₄·5H₂O, 1.6 g/L of MnCl₂·.4H₂O, 0.3 g/L of ZnSO₄·.7H₂O.

The main culture was incubated at 30° C. and 140 rpm for 24 h in an incubator shaker (Infors). After 24 h, 1 ml samples were taken and the cell density OD₆₀₀/ml (optical density of the main culture, measured photometrically at 600 nm), measured using a Genesys™ 10S UV/visible spectrophotometer from Thermo Scientific™, and the content of hypotaurine and taurine determined by HPLC. The content determined by HPLC of hypotaurine in the culture supernatant was 157.3 mg/L for E. coli K12 W3110 x pCys-CDOrn-CSADhs (cell density OD₆₀₀/ml of culture: 5.3/ml). The taurine content in the culture supernatant was 52.8 mg/L.

For E. coli K12 W3110-ppsA-MHI x pCys-CDOrn-CSADhs (cell density OD₆₀₀/ml of culture: 7.1/ml), the content determined by HPLC of hypotaurine in the culture supernatant was 1059.2 mg/L and the content of taurine 304.1 mg/L.

For the two comparison strains E. coli K12 W3110 x pCys (cell density OD₆₀₀/ml of culture: 6.1/mL) and E. coli K12 W3110-ppsA-MHI x pCys (cell density OD₆₀₀/ml of culture: 7.5/ml) neither hypotaurine nor taurine could be detected.

### Example 6

### Oxidation of Hypotaurine FROM the Shake-Flask Culture to Taurine

10 ml each of the batches from the shake-flask culture of E. coli W3110 x pCys-CDOrn-CSADhs and E. coli W3110-ppsA-MHI x pCys-CDOrn-CSADhs (example 5) was centrifuged at 4000 rpm for 10 min (Heraeus™ Megafuge® 1.0 R) and 9.4 ml of the respective supernatant transferred to a 100 ml conical flask and adjusted to pH 5.5 with 0.7 M NaOH. To this were added 0.5 ml of a 200 g/L solution of glucose in H₂O (end concentration in the mixture 10 g/L) and 100 μl of a 1 U/μl stock solution of GOX from Aspergillus niger (Sigma-Aldrich) in 100 mM Na acetate pH 5.5 (end concentration 10 U/ml) and the batches (volume 10 ml) were incubated at 30° C. and 140 rpm in an incubator shaker (Infors). At the start and 2 h after the start of the incubation, 1 ml aliquots of each batch were taken, incubated at 80° C. for 5 min, and centrifuged at 13 000 rpm for 5 min (Heraeus™ Fresco™ 21 centrifuge) and the supernatant analyzed by HPLC.

As summarized in Table 6, the hypotaurine present in the shake-flask culture (example 5)—157.3 mg/L for strain W3110 x pCys-CDOrn-CSADhs and 1059.2 mg/L for strain W3110-ppsA-MHI x pCys-CDOrn-CSADhs—was completely consumed, with the formation of taurine in a concentration of 212.8 mg/L and 1355.6 mg/L respectively. The molar yield was determined taking into account the different molecular weights (109.2 g/mol for hypotaurine, 125.2 g/mol for taurine). As indicated in Table 6, for the strain W3110 x pCys-CDOrn-CSADhs, the combined content of hypotaurine (1.4 mM) and taurine (0.4 mM) at the start of the reaction was 1.8 mM. 2 h after the start of the reaction, the taurine content was 1.7 mM and hypotaurine was no longer detectable. The molar yield of taurine from the enzymatic oxidation of a hypotaurine/taurine product mixture, i.e. based on the total input of hypotaurine and taurine (1.4+0.4=1.8 mM) from the shake-flask culture was 94.4%. For the strain W3110-ppsA-MHI x pCys-CDOrn-CSADhs, the combined content of hypotaurine (9.7 mM) and taurine (2.4 mM) at the start of the reaction was 12.1 mM. 2 h after the start of the reaction, the taurine content was 10.8 mM and hypotaurine was no longer detectable. The molar yield of taurine from the enzymatic oxidation of a hypotaurine/taurine product mixture, i.e. based on the total input of hypotaurine and taurine (9.7+2.4=12.1 mM) from the shake-flask culture was 89.3%.

TABLE 6
Oxidation of hypotaurine from the shake-flask culture of the strains
E. coli K12 W3110 × pCys-CDOrn-CSADhs and E. coli K12
W3110-ppsA-MHI × pCys-CDOrn-CSADhs to taurine after
incubating with GOX for 2 h in the presence of glucose

| Hypotaurine | | Taurine | |
|---|---|---|---|
| mg/L | mM | mg/L | mM |

W3110 × pCys-CDOrn-CSADhs:

| | | | | |
|---|---|---|---|---|
| 0 h | 157.3 | 1.4 | 52.8 | 0.4 |
| 2 h | 0.0 | 0.0 | 212.8 | 1.7 |

W3110-ppsA-MHI × pCys-CDOrn-CSADhs:

| | | | | |
|---|---|---|---|---|
| 0 h | 1059.2 | 9.7 | 304.1 | 2.4 |
| 2 h | 0.0 | 0.0 | 1355.6 | 10.8 |

1-14. (canceled)

15. A process for the enzymatic oxidation of sulfinic acids of formula H₂N—CH(R)—CH₂—SO₂H to sulfonic acids of formula H₂N—CH(R)—CH₂—SO₃H with an enzyme selected from the class of H₂O₂-generating oxidases in the presence of the substrate of said enzyme.

16. The process as claimed in claim 15, wherein the H₂O₂-generating oxidase is alcohol oxidase and that the substrate present is a primary alcohol.

17. The process as claimed in claim 15, wherein the H₂O₂-generating oxidase is glucose oxidase and that the substrate present is glucose.

18. The process as claimed in one or more of claims 15, wherein the sulfinic acid is aminoalkyl sulfinic acid and that the sulfonic acid is aminoalkyl sulfonic acid.

19. The process as claimed in claim 15, wherein the concentration of the sulfinic acid in the batch is at least 1 g/l.

20. The process as claimed in claim 15, wherein the sulfinic acid is 2-aminoethanesulfinic acid (hypotaurine) and that the sulfonic acid formed is 2-aminoethanesulfonic acid (taurine).

21. The process as claimed in claim 15, wherein the sulfonic acid is isolated from the reaction batch.

22. The process as claimed in claim 20, wherein the hypotaurine is hypotaurine from fermentative production.

23. The process as claimed in claim 22, wherein the hypotaurine originates from bacterial production by way of using a bacterial production strain.

24. The process as claimed in claim 23, wherein the bacterial production strain is a strain of the species Escherichia coli.

25. The process as claimed in claim 23, wherein the production strain is a strain having a deregulated cysteine biosynthetic pathway.

26. The process as claimed in claim 23, wherein the production strain is one of the strains E. coli W3110 x pCys-CDOrn-CSADhs or W3110-ppsA-MHI x pCys-CDOrn-CSADhs.

27. The process as claimed in claim 15, wherein the sulfinic acid is cysteine sulfinic acid and that the sulfonic acid formed is cysteic acid.

28. The process as claimed in claim 15, wherein the molar yield of sulfonic acid of formula H₂N—CH(R)—CH₂—SO₃H based on the total molar concentration of sulfinic acid of formula H₂N—CH(R)—CH₂—SO₂H and sulfonic acid of formula H₂N—CH(R)—CH₂—SO₃H used in this reaction is at least 60%.
